(19) United States
(12) Patent Application Publication
(10) Pub. No.: US 2025/0385015 A1
(43) Pub. Date: Dec. 18, 2025

(54) SYSTEM AND METHOD FOR PROVIDING ELECTROCARDIOGRAM READING SERVICE

(71) Applicant: MEDICAL Al CO., LTD., Seoul (KR)

(72) Inventor: Joonmyoung KWON, Seoul (KR)

(21) Appl. No.: 18/875,327

(22) PCT Filed: May 4, 2023
(86) PCT No.: PCT/KR2023/006146

§ 371 Date: Dec. 16, 2024

(30) Foreign Application Priority Data
Jun. 28, 2022 (KR) 10-2022-0079030
Apr. 12, 2023 (KR) 10-2023-0048239

(51) Int. Cl. G16H 80/00; G06F 21/62; G16H 10/60; G16H 15/00; G16H 50/20

(52) U.S. Cl. CPC G16H 80/00; G06F 21/6254; G16H 10/60; G16H 15/00; G16H 50/20

(57) ABSTRACT

The present disclosure is directed to a system and method for providing an electrocardiogram reading service. The system may include: a user terminal configured to generate electrocardiogram data for a user based on user input, to make an electrocardiogram reading request, and to view electrocardiogram reading result data for the electrocardiogram data; a first server configured to receive the electrocardiogram data, to generate de-identification information by using a de-identification code value for user de-identification processing for the electrocardiogram data, and to provide collaboration request data including the generated de-identification information and the electrocardiogram data; and a second server configured to receive the collaboration request data, and to generate electrocardiogram reading result data for the electrocardiogram data based on the collaboration request data by using a pre-trained neural network model.

### TECHNICAL FIELD

The present disclosure relates to a system for providing an electrocardiogram reading service, and more particularly, to a system that provides an electrocardiogram reading service based on electrocardiograms by using a neural network model.

### BACKGROUND ART

Electrocardiograms (ECGs) are signals that are used to determine the presence or absence of disease by checking for abnormalities in a conduction system from the heart to electrodes through the measurement of electrical signals generated in the heart.

The heartbeat, which is the cause of the generation of an electrocardiogram, is performed in such a manner that an impulse that originates from the sinus node located in the right atrium first depolarizes the right and left atria, and, after a brief delay in the atrioventricular node, activates the ventricles.

The right ventricle, which has the fastest septum and thin walls, activates before the left ventricle, which has thick walls. The depolarization waves transferred to the Purkinje fibers spread from the endocardium to the epicardium like wavefronts in the myocardium, thus causing ventricular contraction. Electrical impulses are normally conducted through the heart, and thus the heart contracts approximately 60 to 100 times per minute. Each contraction is represented by heart rate per beat.

Such electrocardiograms can be detected through bipolar leads, which record the potential differences between two portions, and unipolar leads, which record the potentials of the portions to which electrodes are attached. Methods of measuring electrocardiograms include standard limb leads, which are bipolar leads, unipolar limb leads, which are unipolar leads, and precordial leads, which are unipolar leads.

The electrical activity period of the heart is basically divided into atrial depolarization, ventricular depolarization, and ventricular repolarization stages. These individual stages are reflected in the shapes of several waves called P, Q, R, S, and T waves, as shown in FIG. 1.

The electrical activity of the heart can be considered to be normal only when these waves have standard shapes. In order to determine whether these waves have standard shapes, it is necessary to check whether characteristics, such as the times for which the individual waves are maintained, the intervals between the individual waves, the amplitudes of the individual waves, and kurtosis, are within normal ranges.

Such an electrocardiogram is measured with an expensive measurement device and used as an auxiliary tool to measure the health condition of a patient. In general, the electrocardiogram measurement device only displays measurement results, and diagnosis is entirely the responsibility of a doctor.

In particular, a 24-hour electrocardiogram test is a test that measures electrocardiogram changes when a predetermined period of time (for example, about 20 hours) has elapsed after the attachment of a small cassette-sized measurement device to a user's body and thus an electrocardiogram test has been completed. This 24-hour electrocardiogram test is a test for diagnosing heart disease by checking whether symptoms such as dizziness, fainting, palpitations, and chest pain that occur in daily life are related to arrhythmia on electrocardiograms, but there is an inconvenience in that a user needs to personally visit a test room (for example, a hospital) twice to attach and detach the device during the test. In reality, there is the difficulty of having to visit a hospital multiple times to find intermittent abnormal heart signals through an electrocardiogram test, and there is a problem in that patients with mild symptoms or patient receiving follow-up care due to past illnesses need to spend a lot of time and effort on hospital visits.

Currently, research is continuing to rapidly and accurately diagnose diseases based on electrocardiograms by using artificial intelligence in order to reduce dependence on doctors. Furthermore, with the development of wearable type self-electrocardiogram measurement devices such as smartwatches, there is a rising possibility of diagnosing and monitoring not only heart diseases but also various other diseases based on electrocardiograms.

Currently, the positive predictive rate using self-electrocardiogram measurement devices is about 5%, which means that 95% of the results of the self-electrocardiogram measurement devices that are read as having heart disease do not have the disease, so that the reliability is significantly low. In this case, the positive predictive rate refers to the probability that a disease is actually present when it was determined that there was the disease.

When an abnormal finding is found in the electrocardiogram reading results of a self-electrocardiogram measurement device, a user needs to visit a hospital in person to check whether there is no disease, which increases the waste of medical expenses due to unnecessary hospital visits. Furthermore, the reliability of electrocardiograms measured in daily life decreases, which can lead to missing opportunities to detect heart disease early and prevent complications.

In addition, self-electrocardiogram measurement devices such as smartwatches are not linked to medical information systems installed in hospitals, so that users need to print out the electrocardiogram measurement results on a large amount of paper or store them on their mobile phones and then request electrocardiogram reading results from medical staff through hospital visits. Accordingly, there is a problem in that medical staff spend a lot of time reading electrocardiograms for a large number of users, resulting in the waste of medical resources.

Therefore, future electrocardiogram examination systems should not only allow users to continuously measure their electrocardiograms in their daily lives, but should also provide a platform that can rapidly and accurately diagnose diseases via pre-trained neural network models by linking the users' electrocardiogram data with medical information systems installed in hospitals.

### DISCLOSURE

### Technical Problem

The present disclosure has been conceived in response to the above-described background art, and is directed to the provision of a system for an electrocardiogram reading service that reads electrocardiograms measured in daily life, provides reading results for use in hospital treatment, allows users to view the reading results in real time, and recommends a visit to a hospital in the event of a critical condition.

However, the objects to be accomplished by the present disclosure are not limited to the object mentioned above, and other objects not mentioned may be clearly understood based on the following description.

### Technical Solution

According to one embodiment of the present disclosure for achieving the above-described object, there is disclosed a system for providing an electrocardiogram reading service. The system includes: a user terminal configured to generate electrocardiogram data for a user based on user input, to make an electrocardiogram reading request, and to view electrocardiogram reading result data for the electrocardiogram data; a first server configured to receive the electrocardiogram data, to generate de-identification information by using a de-identification code value for user de-identification processing for the electrocardiogram data, and to provide collaboration request data including the generated de-identification information and the electrocardiogram data; and a second server configured to receive the collaboration request data, and to generate electrocardiogram reading result data for the electrocardiogram data based on the collaboration request data by using a pre-trained neural network model; and the first server receives the electrocardiogram reading result data from the second server, identifies the user by decrypting the de-identification information using the de-identification code value, and stores the identified user and the electrocardiogram reading result data in association with each other.

Alternatively, the first server provides the electrocardiogram reading result data as a primary reading result to the user terminal, and, in response to a secondary reading request from the user terminal, obtains expert reading information from an expert terminal for expert in-depth reading through communication with the second server and provides a secondary reading result including the expert reading information to the user terminal, and the second server provides a user interface for expert in-depth reading to the expert terminal, obtains expert reading information from the expert terminal, and provides the expert reading information to the first server.

Alternatively, the electrocardiogram reading result data primarily provided to the user terminal includes at least one of whether there is a disease and disease likelihood score related to the disease that are obtained by the neural network model, and the electrocardiogram reading result data provided by the expert in-depth reading includes expert reading information regarding the disease.

Alternatively, the second server obtains the electrocardiogram reading result data generated by the neural network model as a primary reading result, provides a user interface for expert in-depth reading to the expert terminal, obtains expert reading information as a secondary reading result from the expert terminal, and then generates final electrocardiogram reading result data based on the primary reading result and the secondary reading result.

Alternatively, the second server determines whether the electrocardiogram data included in the collaboration request data is readable by the neural network model, provides a user interface for expert in-depth reading to the expert terminal depending on whether the electrocardiogram data is readable, and obtains expert reading information from the expert terminal.

Alternatively, when the electrocardiogram data included in the collaboration request data is not readable by the neural network model, the second server transmits the collaboration request data to the expert terminal, and receives electrocardiogram reading result data generated by the expert terminal.

Alternatively, when the electrocardiogram reading result data includes information indicating that the user is in an emergency state, the second server provides a user interface for expert in-depth reading to the expert terminal, and then receives expert reading information from the expert terminal.

Alternatively, the second server compares a prediction value for the likelihood of a disease included in the electrocardiogram reading result data with a preset threshold, provides a user interface for expert in-depth reading to the expert terminal depending on the result of the comparison, and obtains expert reading information.

Alternatively, when the prediction value is equal to or larger than the preset threshold value, the second server transmits the collaboration request data to the expert terminal, and receives the electrocardiogram reading result data generated by the expert terminal.

Alternatively, the first server assigns user identification information through user authentication during the initial connection process of the user terminal, and generates the de-identification information by de-identifying user identification information in such a manner as to apply a different de-identification code value for each user or each user group.

Alternatively, when the electrocardiogram reading result data includes a prediction result for a preset electrocardiogram abnormality diagnosis condition, the second server adds warning flag data adapted to provide notification of an electrocardiogram abnormality state to the electrocardiogram reading result data.

Alternatively, the electrocardiogram abnormality diagnosis condition means that there is obtained an abnormal electrocardiogram that deviates from a preset normal reference based on an electrocardiogram characteristic.

Alternatively, when the electrocardiogram reading result data with the warning flag data added thereto is received, the first server provides a notification service for the electrocardiogram reading result data to the user terminal.

Alternatively, the collaboration request data further includes at least one of biological information and electrocardiogram measurement time, and the electrocardiogram reading result data includes the de-identification information and the electrocardiogram reading information.

Meanwhile, according to one embodiment of the present disclosure, there is provided a method of providing an electrocardiogram reading service, the method being performed by a computing device including at least one processor, the method including: generating, by a user terminal, electrocardiogram data for a user based on user input, and making, by the user terminal, an electrocardiogram reading request to a first server; receiving, by the first server, the electrocardiogram data from the user terminal, generating, by the first server, de-identification information by using a de-identification code value for user de-identification processing for the electrocardiogram data, generating, by the first server, collaboration request data including the generated de-identification information and the electrocardiogram data, and transmitting, by the first server, the collaboration request data to a second server; and receiving, by the second server, the collaboration request data from the first server, generating, by the second server, electrocardiogram reading result data for the electrocardiogram data based on the collaboration request data by using a pre-trained neural network model, and transmitting, by the second server, the electrocardiogram reading result data to the first server; wherein the first server receives the electrocardiogram reading result data from the second server, identifies the user by decrypting the de-identification information using the de-identification code value, and stores the identified user and the electrocardiogram reading result data in association with each other so that the electrocardiogram reading result data for the user is viewed in the user terminal.

### Advantageous Effects

The system for providing an electrocardiogram reading service according to one embodiment of the present disclosure may detect heart disease early by linking the electrocardiogram data, transmitted from a user terminal, with a medical information system installed in a hospital and rapidly and accurately reading de-identified electrocardiogram data using the neural network model. Furthermore, expert in-depth reading is performed on unreadable electrocardiogram data, so that the risk of medical accidents can be reduced and the waste of medical resources required for reading the electrocardiogram data measured by a self-electrocardiogram measurement device can be reduced.

In addition, the system providing an electrocardiogram reading service according to one embodiment of the present disclosure may share an electrocardiogram reading result with an institution designated by a user via a medical information system installed in a hospital, and thus provides the effect of being able to link the electrocardiogram reading service with various medical services and healthcare services.

### DESCRIPTION OF DRAWINGS

FIG. 1 is a diagram showing an electrocardiogram signal according to the present disclosure;

FIG. 2 is a block diagram of a computing device according to one embodiment of the present disclosure;

FIG. 3 block is a diagram illustrating the configuration of a system for providing an electrocardiogram reading service according to one embodiment of the present disclosure;

FIG. 4 is an exemplary diagram illustrating a service app execution screen on a user terminal according to one embodiment of the present disclosure;

FIG. 5 is a flowchart showing a method of providing an electrocardiogram reading service according to one embodiment of the present disclosure; and

FIG. 6 is an exemplary diagram illustrating electrocardiogram reading information according to one embodiment of the present disclosure.

### MODE FOR INVENTION

Embodiments of the present disclosure will be described in detail below with reference to the accompanying drawings so that those having ordinary skill in the art of the present disclosure (hereinafter referred to as those skilled in the art) can easily implement the present disclosure. The embodiments presented in the present disclosure are provided to enable those skilled in the art to use or practice the content of the present disclosure. Accordingly, various modifications to embodiments of the present disclosure will be apparent to those skilled in the art. That is, the present disclosure may be implemented in various different forms and is not limited to the following embodiments.

The same or similar reference numerals denote the same or similar components throughout the specification of the present disclosure. Additionally, in order to clearly describe the present disclosure, reference numerals for parts that are not related to the description of the present disclosure may be omitted in the drawings.

The term “or” used herein is intended not to mean an exclusive “or” but to mean an inclusive “or.” That is, unless otherwise specified herein or the meaning is not clear from the context, the clause “X uses A or B” should be understood to mean one of the natural inclusive substitutions. For example, unless otherwise specified herein or the meaning is not clear from the context, the clause “X uses A or B” may be interpreted as any one of a case where X uses A, a case where X uses B, and a case where X uses both A and B.

The term “and/or” used herein should be understood to refer to and include all possible combinations of one or more of listed related concepts.

The terms “include” and/or “including” used herein should be understood to mean that specific features and/or components are present. However, the terms “include” and/or “including” should be understood as not excluding the presence or addition of one or more other features, one or more other components, and/or combinations thereof.

Unless otherwise specified herein or unless the context clearly indicates a singular form, the singular form should generally be construed to include “one or more.”

The term “n-th (n is a natural number)” used herein can be understood as an expression used to distinguish the components of the present disclosure according to a predetermined criterion such as a functional perspective, a structural perspective, or the convenience of description. For example, in the present disclosure, components performing different functional roles may be distinguished as a first component or a second component. However, components that are substantially the same within the technical spirit of the present disclosure but should be distinguished for the convenience of description may also be distinguished as a first component or a second component.

The term “obtaining” used herein can be understood to mean not only receiving data over a wired/wireless communication network connecting with an external device or a system, but also generating data in an on-device form.

Meanwhile, the term “module” or “unit” used herein may be understood as a term referring to an independent functional unit processing computing resources, such as a computer-related entity, firmware, software or part thereof, hardware or part thereof, or a combination of software and hardware. In this case, the “module” or “unit” may be a unit composed of a single component, or may be a unit expressed as a combination or set of multiple components. For example, in the narrow sense, the term “module” or “unit” may refer to a hardware component or set of components of a computing device, an application program performing a specific function of software, a procedure implemented through the execution of software, a set of instructions for the execution of a program, or the like. Additionally, in the broad sense, the term “module” or “unit” may refer to a computing device itself constituting part of a system, an application running on the computing device, or the like. However, the above-described concepts are only examples, and the concept of “module” or “unit” may be defined in various manners within a range understandable to those skilled in the art based on the content of the present disclosure.

The term “model” used herein may be understood as a system implemented using mathematical concepts and language to solve a specific problem, a set of software units intended to solve a specific problem, or an abstract model for a process intended to solve a specific problem. For example, a neural network “model” may refer to an overall system implemented as a neural network that is provided with problem-solving capabilities through training. In this case, the neural network may be provided with problem-solving capabilities by optimizing parameters connecting nodes or neurons s through training. The neural network “model” may include a single neural network, or a neural network set in which multiple neural networks are combined together.

The term “block” used herein may be understood as a set of components classified based on various criteria such as type, function, etc. Accordingly, the components classified as each “block” may be changed in various manners depending on the criteria. For example, a neural network “block” may be understood as a set of neural networks including one or more neural networks. In this case, it can be assumed that the neural networks included in the neural network “block” perform the same specific operations. The foregoing descriptions of the terms are intended to help to understand the present disclosure. Accordingly, it should be noted that unless the above-described terms are explicitly described as limiting the content of the present disclosure, the terms in the content of the present disclosure are not used in the sense of limiting the technical spirit of the present disclosure.

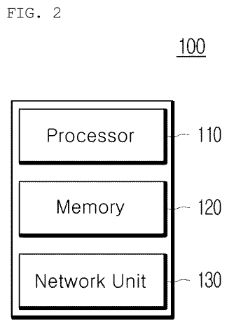

FIG. 2 is a block diagram of a computing device according to one embodiment of the present disclosure.

A computing device 100 according to one embodiment of the present disclosure may be a hardware device or part of a hardware device that performs the comprehensive processing and calculation of data, or may be a software-based computing environment that is connected to a communication network. For example, the computing device 100 may be a server that performs an intensive data processing function and shares resources, or may be a client that shares resources through interaction with a server. Furthermore, the computing device 100 may be a cloud system in which a plurality of servers and clients interact with each other and comprehensively process data. Since the above descriptions are only examples related to the type of computing device 100, the type of computing device 100 may be configured in various manners within a range understandable to those skilled in the art based on the content of the present disclosure.

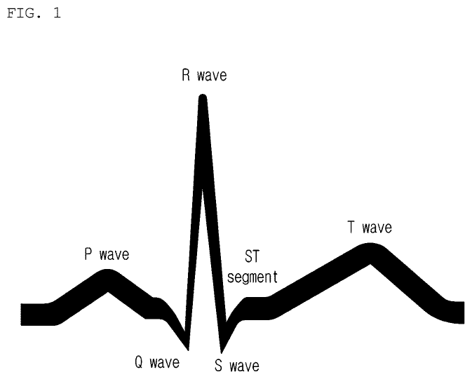

Referring to FIG. 2, the computing device 100 according to one embodiment of the present disclosure may include a processor 110, memory 120, and a network unit 130. However, FIG. 1 shows only an example, and the computing device 100 may include other components for implementing a computing environment. Furthermore, only some of the components disclosed above may be included in the computing device 100.

The processor 110 according to one embodiment of the present disclosure may be understood as a constituent unit including hardware and/or software for performing computing operation. For example, the processor 110 may read a computer program and perform data processing for machine learning. The processor 110 may process computational processes such as the processing of input data for machine learning, the extraction of features for machine learning, and the calculation of errors based on backpropagation. The processor 110 for performing such data processing may include a central processing unit (CPU), a general purpose graphics processing unit (GPGPU), a tensor processing unit (TPU), an application specific integrated circuit (ASIC), or a field programmable gate array (FPGA). Since the types of processor 110 described above are only examples, the type of processor 110 may be configured in various manners within a range understandable to those skilled in the art based on the content of the present disclosure.

The processor 110 may train a neural network model that diagnoses heart disease based on electrocardiogram data. For example, the processor 110 may train the neural network model to estimate arrhythmia and other heart diseases based information, including on biological information such as gender, age, weight, height, and/or the like, together with electrocardiogram data. More specifically, the processor 110 may train the neural network model so that the neural network model can detect changes in electrocardiograms attributable to arrhythmia or another heart disease by inputting electrocardiogram data and various types of biological information to the neural network model. In this case, the neural network model may be trained based on an electrocardiogram dataset including the features extracted from electrocardiogram data and diagnostic data for arrhythmia and other heart diseases. The processor 110 may perform the operation of representing at least one neural network block included in the neural network model during the process of training the neural network model.

The processor 110 may estimate electrocardiogram reading result data based on the electrocardiogram data input by a user by using the neural network model generated through the above-described training process. The processor 110 may generate inference data representing the result of estimating the likelihood of a heart disease by inputting electrocardiogram data and biological information, including information such as gender, age, weight, height, and/or the like, to the neural network model trained through the above-described process. For example, the processor 110 may predict the presence or absence of arrhythmia or another heart disease, the degree of progression thereof, and/or the like by inputting electrocardiogram data to the trained neural network model.

In addition to the examples described above, the types of medical data and the output of the neural network model may be configured in various manners within a range understandable to those skilled in the art based on the content of the present disclosure.

The memory 120 according to one embodiment of the present disclosure may be understood as a constituent unit including hardware and/or software for storing and managing data that is processed in the computing device 100. That is, the memory 120 may store any type of data generated or determined by the processor 110 and any type of data received by the network unit 130. For example, the memory 120 may include at least one type of storage medium of a flash memory type, hard disk type, multimedia card micro type, and card type memory, random access memory (RAM), static random access memory (SRAM), read-only memory (ROM), electrically erasable programmable read-only memory (EEPROM), programmable read-only memory (PROM), magnetic memory, a magnetic disk, and an optical disk. Furthermore, the memory 120 may include a database system that controls and manages data in a predetermined system. Since the types of memory 120 described above are only examples, the type of memory 120 may be configured in various manners within a range understandable to those skilled in the art based on the content of the present disclosure.

The memory 120 may structure, organize, and manage data necessary for the processor 110 to perform computation, the combination of data, and program codes executable on the processor 110. For example, the memory 120 may store medical data received through the network unit 130, which will be described later. The memory 120 may store program codes configured to operate the neural network model to receive medical data and perform learning, program codes configured to operate the neural network model to receive medical data and perform inference in accordance with the purpose of use of the computing device 100, processed data generated as program codes are executed, etc.

The network unit 130 according to one embodiment of the present disclosure may be understood as a constituent unit that transmits and receives data through any type of known wired/wireless communication system. For example, the network unit 130 may perform data transmission and reception using a wired/wireless communication system such as a local area network (LAN), a wideband code division multiple access (WCDMA) network, a long term evolution (LTE) network, the wireless broadband Internet (WiBro), a 5th generation mobile communication (5G) network, a ultra wide-band wireless communication network, a ZigBee network, a radio frequency (RF) communication network, a wireless LAN, a wireless fidelity network, a near field communication (NFC) network, or a Bluetooth network. Since the above-described communication systems are only examples, the wired/wireless communication system for the data transmission and reception of the network unit 130 may be applied in various manners other than the above-described examples.

The network unit 130 may receive data necessary for the processor 110 to perform computation through wired/wireless communication with any system or client or the like. Furthermore, the network unit 130 may transmit data generated through the computation of the processor 110 through wired/wireless communication with any system or client or the like. For example, the network unit 130 may receive medical data through communication with a database within a hospital environment, a cloud server configured to perform tasks such as the standardization of medical data, a computing device, or the like. The network unit 130 may transmit the output data of the neural network model, intermediate data and processed data acquired from the computation process of the processor 110, etc. through communication with the above-described database, server, or computing device.

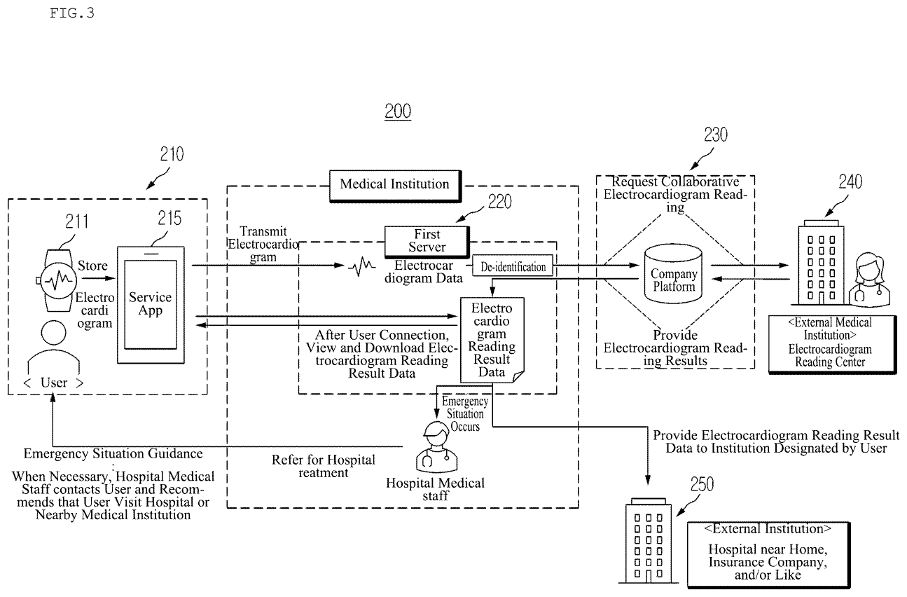

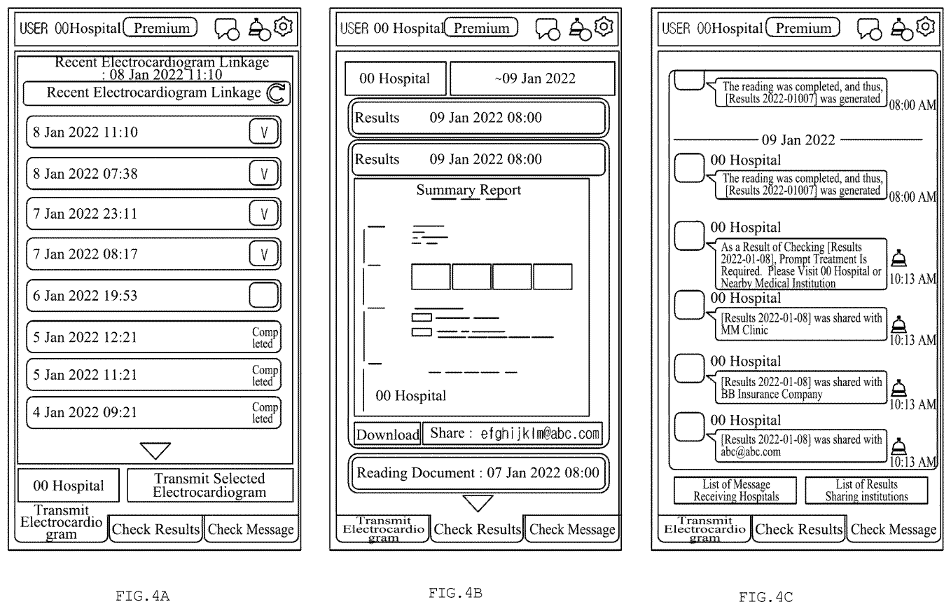

FIG. 3 is a block diagram illustrating the configuration of a system for providing an electrocardiogram reading service according to one embodiment of the present disclosure, and FIG. 4 is an exemplary diagram illustrating a service app execution screen on a user terminal according to one embodiment of the present disclosure.

The system 200 for providing an electrocardiogram reading service includes, but is not limited to, a user terminal 210, a first server 220, a second server 230, and an expert terminal 240.

The user terminal 210 may generate electrocardiogram data for a user based on user input, may make an electrocardiogram reading request to the first server 220, and may view electrocardiogram reading result data for the electrocardiogram data through the first server 220.

This user terminal 210 may include a wearable device 111 that is worn on a user's body and can measure and collect various health indicators such as heart rate, body fat percentage, blood pressure, and/or the like, and a smart terminal 115 that is connected thereto. The wearable device 111 may include an electronic accessory and smartwatch that have been approved as medical devices by the Ministry of Food and Drug Safety to measure daily life electrocardiograms, and the smart terminal 115 may include a mobile phone, a smart phone, a laptop computer, a digital broadcasting terminal, a personal digital assistant (PDA), a portable multimedia player (PMP), a navigation system, a slate PC, a tablet PC, an ultrabook, and the like. In this case, the daily life electrocardiograms may mean electrocardiograms that are measured by oneself outside a hospital by using the wearable device 111 approved as a medical device by the Ministry of Food and Drug Safety.

Meanwhile, although a case where electrocardiograms are measured by a single-lead method using a wearable device 211 such as a wristwatch or patch is described as an example in the present disclosure, electrocardiograms may be measured by various electrode combinations such as a 12-lead method, a 6-lead method, and the like. It is also desirable that the electrocardiogram measurement time is increased or decreased and then set according to the signal to be obtained.

The smart terminal 215 may execute a service app related to an electrocardiogram reading service and perform functions such as data selection, transmission, viewing, and downloading. The user may select the electrocardiogram data measured by the wearable device 211 via the service app executed on the smart terminal 215 and transmit it to the first server 220 (see (a) of FIG. 3). In this case, an ‘electrocardiogram data linkage’ button may link the electrocardiogram data, stored in a designated folder, to the data list of the service app via the app installed on the wearable device 111.

The user terminal 210 may perform the function of connecting with the first server 220 and viewing and downloading electrocardiogram reading result data for the user and transmitting the electrocardiogram reading result data to a designated email address via the service app (see (b) of FIG. 3).

The user terminal 210 may check a notification message, transmitted from the first server 220, via the service app (see (c) of FIG. 3). In this case, a message window output via the service app stores only one-way messages transmitted from the first server 220 to the user terminal 210, and the user may not transmit a message to the first server 220 through the message window. However, the user may select the first server 220 for the reception of a notification message from a list of message receiving hospitals, and may select a fourth server 250 for the sharing of the user's electrocardiogram reading result data from a list of results sharing institutions.

The first server 220 may receive electrocardiogram data from the user terminal 210, may generate de-identification information by encrypting user identification information using a de-identification code value for user de-identification processing for the electrocardiogram data, and may generate collaboration request data including the generated de-identification information and the electrocardiogram data. Furthermore, the first server 220 may receive electrocardiogram reading result data from the second server 230, may identify the user by decrypting the de-identification information of the collaboration request data using the de-identification code value, and may store the identified user and the electrocardiogram reading result data in association with each other. In this case, the first server 220 may completely remove the identifiability of personal information by using a one-way encryption method without sharing the de-identification code value with the user terminal or another server.

This first server 220 may be a server that provides medical information through the user terminal 210 in conjunction with a medical information system installed in a medical institution that provides medical services, such as a hospital, public health center, or medical examination center.

The first server 220 may provide user identification information by authenticating the user during the initial connection process of the user terminal 210, and may generate de-identification information through an encryption process by applying the already generated de-identification code value of the user terminal 210 to user identification information. The first server 220 may apply a different de-identification code value for each user or each institution of a user.

In this case, the user identification information may be patient information. In the case where there is no patient information for the user terminal in a medical institution linked to the first server 220 when the user terminal 210 initially connects with the first server 220, the first server 220 may generate user identification information with the user's consent.

The second server 230 may receive the collaboration request data from the first server 220, may generate electrocardiogram reading result data for the electrocardiogram data based on the collaboration request data by using a pre-trained neural network model, and may transmit the electrocardiogram reading result data to the first server 220.

The second server 230 may provide a service app to the user terminal 210 through an app registration procedure in response to a request from the user terminal 210, and may provide an app management service, including follow-up measures such as updates to the service app, according to a preset app management policy.

During the app registration procedure, the second server 230 may provide a service app to a user terminal 210 that has agreed to provide personal information and data in order to use the electrocardiogram reading service. Whenever the user terminal 210 adds a sharing institution for the sharing of the electrocardiogram reading result data, the second server 230 may individually receive consent for the provision of data via the service app and then perform the transmission of data.

The second 230 may determine whether the electrocardiogram data included in the collaborative request data is readable by the neural network model, may provide a user interface for expert in-depth reading to the expert terminal 240 based on whether the electrocardiogram data is readable, and may obtain expert reading information through communication with the expert terminal 240.

When the electrocardiogram data is readable by the neural network model, the second server 230 may generate electrocardiogram reading result data by inputting the electrocardiogram data to the pre-trained neural network model. In this case, the case where the electrocardiogram data is readable may be understood as a state in which the minimum information on which the neural network model can perform interpretation (or inference) is included in the electrocardiogram data. Furthermore, the electrocardiogram reading result data may include information related to the diagnosis of a disease, such as whether a measurement target for the electrocardiogram data has a disease, the type of disease, the degree of progression of the disease, a disease likelihood score, and/or the like.

The second server 230 may request collaboration by transmitting collaboration request data to the expert terminal 240 when electrocardiogram reading is impossible due to noise signals included in the electrocardiogram data or error signals caused by inaccurate measurement. The expert terminal 240 may be an electrocardiogram reading center that can collaborate with an external expert and provide expert in-depth reading services. That is, when the electrocardiogram data is difficult for the neural network model to interpret or make an inference from, the second server 230 may rapidly transmit collaboration request data, including the electrocardiogram data, to the expert terminal 240 that provides a medical expert's diagnostic service. When reading is impossible even via the expert terminal 240, the second server 230 may transmit guidance on the re-measurement of electrocardiogram data to the user terminal 210 based on feedback from the expert terminal 240. The guidance on the re-measurement of electrocardiogram data may be generated by the second server 230 in accordance with the type of the user terminal 210, and may include information about an electrocardiogram measurement method, measurement time, and/or the like.

Meanwhile, the second server 230 may communicate with the expert terminal 240 to automatically perform expert reading based on the result of the neural network model. That is, when the reading result of the electrocardiogram data by the neural network model includes information indicating that the state is ‘emergency’, the second server 230 may automatically perform expert in-depth reading via the expert terminal 240 even without the user's consent. Alternatively, when the prediction value for the likelihood of a disease in the reading result of the electrocardiogram data by the neural network model is equal to or higher than a preset threshold, the second server 230 may determine the patient's condition to be an emergency level and automatically perform expert in-depth reading via the expert terminal 240 even without the user's consent.

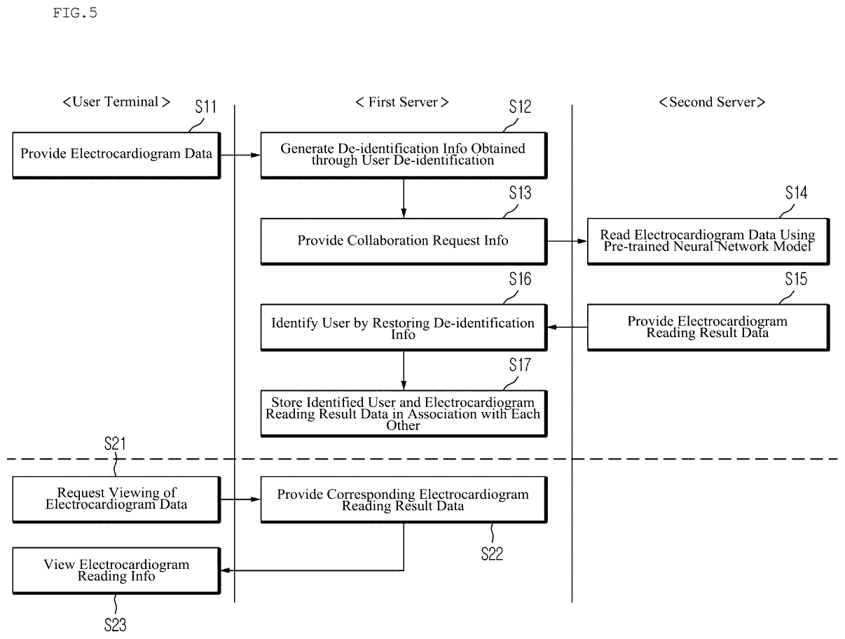

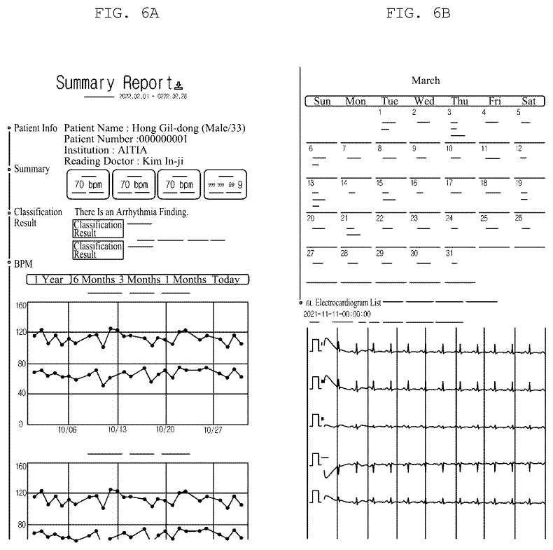

FIG. 5 is a flowchart showing a method of providing an electrocardiogram reading service according to one embodiment of the present disclosure, and FIG. 6 is an exemplary diagram illustrating electrocardiogram reading information according embodiment of the present disclosure.

Referring to FIG. 5, there is shown a method of providing an electrocardiogram reading service that is performed by a computing device including at least one processor, in which step S11 of obtaining, by the first server 220, electrocardiogram data from the user terminal 210 may be performed first.

The electrocardiogram data may be directly obtained through measurement via the wearable device 211, or may be obtained from the smart terminal 215 connected to the wearable device 211 through network communication. The first server 220 may store the electrocardiogram data transmitted from the user terminal 210, and may generate collaboration request data by associating the electrocardiogram data with de-identification information for user de-identification processing in response to an electrocardiogram reading request from a user in steps S12 and S13. In this case, the collaboration request data includes the de-identification information and the electrocardiogram data, and may further include biological information and electrocardiogram measurement time. The electrocardiogram measurement time may be data required to match an electrocardiogram measured at a specific point in time with a specific reading result when an electrocardiogram is measured for the same person at least once. Accordingly, the electrocardiogram measurement time may be included in either the de-identification information or the electrocardiogram data.

The first server 220 may transmit collaboration the consultation request data to the second server 230, and the second server 230 may estimate electrocardiogram reading information for the user of the electrocardiogram data based on the collaboration request data by using the pre-trained neural network model and provide electrocardiogram reading result data including de-identification information and electrocardiogram reading information to the first server 220, in steps S14 and S15.

An electrocardiogram is a unique signal for each individual because it differs depending on gender, age, and the location and size of the heart. Accordingly, there may be included the step in which the second server 230 may estimate electrocardiogram reading information for the user of the electrocardiogram inputting biological information, including at least one of age, gender, weight, and height, together with the electrocardiogram data to the neural network model.

In this case, the neural network model may be trained on electrocardiogram data on a per-feature basis by using deep learning algorithms, and may derive electrocardiogram reading information, including the diagnostic name of a heart disease, by using the trained model. Furthermore, the neural network model may be trained based on the correlations between left ventricular systolic dysfunction and changes in characteristics such as electrocardiogram, gender, age, weight, height, and/or the like. More specifically, the neural network model may be trained based on the correlations between various factors in a training dataset including electrocardiograms and heart disease diagnosis results.

The neural network model may be trained based on the electrocardiograms obtained from electrodes of an electrocardiogram measurement device connected to the human body and measured with 12 leads. As an example, an electrocardiogram may be measured with 12 leads for ten seconds in length, and may be stored as 500 points per second. Additionally, the neural network model may be trained based on partial information obtained by extracting only 6-limb lead electrocardiograms and single-lead (lead I) electrocardiograms from 12-lead electrocardiograms.

More specifically, the neural network model may receive electrocardiogram data and output electrocardiogram reading information. The neural network model may include at least one convolutional neural network (CNN), a batch normalization, and an ReLU activation function layer, and may further include a dropout layer. The neural network model may include a fully connected layer via which biological information such as age, gender, height, weight, and/or the like is input as auxiliary information.

The neural network model may include neural networks corresponding to the plurality of leads of electrocardiogram data, respectively. That is, the neural network model may include individual neural networks to which electrocardiograms measured with individual leads are input.

Meanwhile, the structure of the neural network model and type of the neural network described above are only examples, and thus, the neural network model according to one embodiment of the present invention may be configured in various manners based on the examples described above.

Referring again to FIG. 5, the first server 220 may identify the user by decrypting the de-identifying information using the de-identification code value in the electrocardiogram reading result data received from the second server 230 and store the identified user and the electrocardiogram reading result data in association with each other in steps S16 and S17. As shown in FIG. 6, the first server 220 may store the electrocardiogram reading result data for the electrocardiogram reading information including the identified user identification information (e.g., patient information) and the diagnosis of the electrocardiogram data, whether the patient has a disease such as a heart disease finding, and a disease likelihood score.

When the first server 220 receives the electrocardiogram reading result data from the second server 230, it may transmit a notification message via the service app of the user terminal 210 within a preset time.

The user terminal 210 may connect with the first server 220, may request an inquiry of its electrocardiogram reading result data, and may view the electrocardiogram reading information provided by the first server 220 in steps S21, S22, and S23.

The first server 220 may transmit a checking message for consent to the reception of secondary reading so that the user can select whether to receive expert reading while providing the user terminal 210 with the electrocardiogram reading information as a primary reading result. The first server 220 may include information about the cost required for a secondary reading procedure in the checking message and provide a resulting checking message.

For the user terminal 210 that has agreed to receive secondary reading, the first server 220 may notify the second server 230 of a secondary reading request from the user terminal 210, and the second server 230 may provide a user interface to the expert terminal 240 so that expert reading information can be obtained from the expert terminal 240. The second server 230 communicates with the first server 220 and provides expert reading information for the electrocardiogram data, obtained from the expert terminal 240, to the first server 220. In this case, the second server 230 may transmit collaboration request data including de-identification information and electrocardiogram data for the user terminal 210 to the expert terminal 240, and the first server 220 may provide the expert reading information, received via the second server 230, as a secondary reading result to the user terminal 210. Alternatively, for the user terminal 210 that has agreed to receive secondary reading, the first server 220 may provide de-identification information together with the primary reading result to the expert terminal 240 via the second server 230.

Meanwhile, after the primary reading result has been generated by the neural network model, the second server 230 may obtain expert reading information through communication with the expert terminal 240 so that a secondary reading result is automatically generated by the expert terminal 240. In this case, the second server 230 may perform a prior consent procedure for primary reading and secondary reading when the user terminal 210 initially connects with the second server 230.

The first server 220 may transmit a primary reading result text, such as ‘non-emergency’ or ‘expert reading required,’ as a primary reading result to the user terminal 210, and may provide expert reading information, including detailed descriptions of a disease name and a disease likelihood, as a secondary reading result. For example, the expert reading information may include information such as “This patient has signs of ‘premature atrial contraction.’ This reading result is not the final or definite diagnosis result of the patient, so that it is recommended that you visit a nearby hospital for an accurate examination and treatment.” Meanwhile, when a prediction result for a preset electrocardiogram abnormality diagnosis condition is included in the electrocardiogram reading information estimated via the neural network model, the second server 230 may add warning flag data adapted to provide notification of an electrocardiogram abnormality state to the electrocardiogram reading result data. In this case, the electrocardiogram abnormality diagnosis condition may mean that there is obtained an abnormal electrocardiogram that deviates from a preset normal reference based on an electrocardiogram feature including at least one of the frequency of tachycardia, the length of the QT interval, the directions of deviation of the P wave, the R wave, and the T wave, and the duration of the QRS.

When the first server 220 receives the electrocardiogram reading result data with warning flag data added thereto from the second server 230, it may provide a notification service for the electrocardiogram reading result data to the user terminal 210. For example, the first server 220 may transmit the information of the notification service to the terminal of medical staff or a guide, and the medical staff or the guide may provide the user terminal 210 with a text message or call service recommending a visit to a hospital or medical institution.

When a notification service is provided from the first server 220 to the user terminal 210, the user may select a results sharing institution via the service app and transmit the electrocardiogram reading result data from the first server 220 to the fourth server 250. In this case, the fourth server 250 may be a medical information system that is installed in a medical institution near the user's home, the user's insurance company, or the like.

Vis this system and the reading process performed via the system, users may freely measure electrocardiograms at any time and place they want, and easily monitor their health states. Furthermore, hospitals may prompt users to visit the hospitals as needed, so that there can be set up a medical environment where responses to the onset of disease can be carried out preemptively.

The various embodiments of the present disclosure described above may be combined with one or more additional embodiments, and may be changed within the range understandable to those skilled in the art in light of the above detailed description. The embodiments of the present disclosure should be understood as illustrative but not restrictive in all respects. For example, individual components described as unitary may be implemented in a distributed manner, and similarly, the components described as distributed may also be implemented in a combined form. Accordingly, all changes or modifications derived from the meanings and scopes of the claims of the present disclosure and their equivalents should be construed as being included in the scope of the present disclosure.

1. A system for providing an electrocardiogram reading service, the system comprising:
a user terminal configured to generate electrocardiogram data for a user based on user input, to make an electrocardiogram reading request, and to view electrocardiogram reading result data for the electrocardiogram data;
a first server configured to receive the electrocardiogram data, to generate de-identification information by using a de-identification code value for user de-identification processing for the electrocardiogram data, and to provide collaboration request data including the generated de-identification information and the electrocardiogram data; and
a second server configured to receive the collaboration request data, and to generate electrocardiogram reading result data for the electrocardiogram data based on the collaboration request data by using a pre-trained neural network model;
wherein the first server receives the electrocardiogram reading result data from the second server, identifies the user by decrypting the de-identification information using the de-identification code value, and stores the identified user and the electrocardiogram reading result data in association with each other.

2. The system of claim 1, wherein:
the first server provides the electrocardiogram reading result data as a primary reading result to the user terminal, and, in response to a secondary reading request from the user terminal, obtains expert reading information from an expert terminal for expert in-depth reading through communication with the second server and provides a secondary reading result including the expert reading information to the user terminal; and
the second server provides a user interface for expert in-depth reading to the expert terminal, obtains expert reading information from the expert terminal, and provides the expert reading information to the first server.

3. The system of claim 2, wherein:
the electrocardiogram reading result data primarily provided to the user terminal includes at least one of whether there is a disease and disease likelihood score related to the disease that are obtained by the neural network model; and
the electrocardiogram reading result data provided by the expert in-depth reading includes expert reading information regarding the disease.

4. The system of claim 1, wherein the second server obtains the electrocardiogram reading result data generated by the neural network model as a primary reading result, provides interface for expert in-depth reading to the expert terminal, obtains expert reading information as a secondary reading result from the expert terminal, and then generates final electrocardiogram reading result data based on the primary reading result and the secondary reading result.

5. The system of claim 1, wherein the second server determines whether the electrocardiogram data included in the collaboration request data is readable by the neural network model, provides a user interface for expert in-depth reading to the expert terminal depending on whether the electrocardiogram data is readable, and obtains expert reading information from the expert terminal.

6. The system of claim 5, wherein the second server, when the electrocardiogram data included in the collaboration request data is not readable by the neural network model, transmits the collaboration request data to the expert terminal and receives electrocardiogram reading result data generated by the expert terminal.

7. The system of claim 1, wherein the second server, when the electrocardiogram reading result data includes information indicating that the user is in an emergency state, provides a user interface for expert in-depth reading to the expert terminal and then receives expert reading information from the expert terminal.

8. The system of claim 1, wherein the second server compares a prediction value for a likelihood of a disease included in the electrocardiogram reading result data with a preset threshold, provides a user interface for expert in-depth reading to the expert terminal depending on a result of the comparison, and obtains expert reading information.

9. The system of claim 8, wherein the second server, when the prediction value is equal to or larger than the preset threshold value, transmits the collaboration request data to the expert terminal and receives the electrocardiogram reading result data generated by the expert terminal.

10. The system of claim 1, wherein the first server:
assigns user identification information through user authentication during an initial connection process of the user terminal; and
generates the de-identification information by de-identifying user identification information in such a manner as to apply a different de-identification code value for each user or each user group.

11. The system of claim 1, wherein the second server, when the electrocardiogram reading result data includes a prediction result for a preset electrocardiogram abnormality diagnosis condition, adds warning flag data adapted to provide notification of an electrocardiogram abnormality state to the electrocardiogram reading result data.

12. The system of claim 11, wherein the electrocardiogram abnormality diagnosis condition means that there is obtained an abnormal electrocardiogram that deviates from a preset normal reference based on an electrocardiogram characteristic.

13. The system of claim 11, wherein the first server, when the electrocardiogram reading result data with the warning flag data added thereto is received, provides a notification service for the electrocardiogram reading result data to the user terminal.

14. The system of claim 1, wherein:
the collaboration request data further includes at least one of biological information and electrocardiogram measurement time; and
the electrocardiogram reading result data includes the de-identification information and the electrocardiogram reading information.

15. A method of providing an electrocardiogram reading service, the method being performed by a computing device including at least one processor, the method comprising:
generating, by a user terminal, electrocardiogram data for a user based on user input, and making, by the user terminal, an electrocardiogram reading request to a first server;
receiving, by the first server, the electrocardiogram data from the user terminal, generating, by the first server, de-identification information by using a de-identification code value for user de-identification processing for the electrocardiogram data, generating, by the first server, collaboration request data including the generated de-identification information and the electrocardiogram data, and transmitting, by the first server, the collaboration request data to a second server; and
receiving, by the second server, the collaboration request data from the first server, generating, by the second server, electrocardiogram reading result data for the electrocardiogram data based on the collaboration request data by using a pre-trained neural network model, and transmitting, by the second server, the electrocardiogram reading result data to the first server;
wherein the first server receives the electrocardiogram reading result data from the second server, identifies the user by decrypting the de-identification information using the de-identification code value, and stores the identified user and the electrocardiogram reading result data in association with each other so that the electrocardiogram reading result data for the user is viewed in the user terminal.
